(19) United States
(12) Patent Application Publication
(10) Pub. No.: US 2023/0329360 A1
(43) Pub. Date: Oct. 19, 2023

(54) Lumpectomy Shower Bra Device

(71) Applicant: Theresa Abbazia, Weeki Wachee, FL (US)

(72) Inventor: Theresa Abbazia, Weeki Wachee, FL (US)

(21) Appl. No.: 18/295,393

(22) Filed: Apr. 4, 2023

Related U.S. Application Data
(60) Provisional application No. 63/330,963, filed on Apr. 14, 2022.

(51) Int. Cl. A41C 3/00

(52) U.S. Cl. CPC A41C 3/0064; A61F 2/52

(57) ABSTRACT

The present invention relates to a novel lumpectomy shower bra device. The device is designed for lumpectomy patients to comfortably and independently shower. Specifically, the lumpectomy shower bra device comprises a racer back style with straps over the shoulder, and a closure positioned in the front of the bra. The device comprises soft, lily pad-like cups which supports a user’s breasts and prevents water pressure from hitting the incision site. Further, the bra device is also constructed using a quick-drying material for daily reuse while bathing. Thus, users can utilize both arms while taking a shower or bathing, and can utilize shampoo, conditioner, body scrubs, etc.

### CROSS-REFERENCE TO RELATED APPLICATION

The present application claims priority to, and the benefit of, U.S. Provisional Application No. 63/330,963, which was filed on April 14th, 2022 and is incorporated herein by reference in its entirety.

### FIELD OF THE INVENTION

The present invention relates generally to the field of lumpectomy bras or brassieres. More specifically, the present invention relates to an improved lumpectomy shower bra device that provides users with a modified bra capable of comfortably supporting the breasts while bathing. Accordingly, the present disclosure makes specific reference thereto. Nonetheless, it is to be appreciated that aspects of the present invention are also equally applicable to other like applications, devices and methods of manufacture.

### BACKGROUND

By way of background, each year, thousands of women undergo breast surgery. Of all the surgeries that a woman may experience, breast surgery, and particularly lumpectomy or removal of a tumor and/or mastectomy or removal of a breast or breasts, is the most traumatic. A lumpectomy and/or a mastectomy is the most common surgical procedure performed when a malignant tumor is found. The type of surgery depends upon the staging of the tumor and the user’s preferences. Although many women look for more conservative treatment and less destructive surgery than removal of the breast, lumpectomy and/or mastectomy continues to be the preferred course of treatment to increase the likelihood of destroying the breast cancer. Specifically, mastectomy procedures include modified radical mastectomy (removal of the underlying muscle as well as the breast), simple mastectomy (removal of one breast), bilateral mastectomy (removal of both breasts) and lumpectomy (removal of a portion of the breast).

When the cancer involves the muscle or interpectoral node, substantially more muscle and tissue must be removed. Removal of auxiliary nodes and lymphatic channels predisposes the client to infection and lymphatic obstruction. Edema, an abnormal excess accumulation of fluid in connective tissue, is a frequent occurrence in breast surgeries unless positive steps are taken to prevent it. The body develops a collateral lymphatic drainage system usually within three to four weeks postoperatively. In the interim, lymphatic fluid production must be drained to prevent infection and promote healing. Post operative drainage tubes are placed in the body to aid in lymphatic fluid drainage. Drainage tube systems are used not only for mastectomies but also for breast reconstructive surgery as well as for less radical surgeries for breast enlargement or breast reduction.

Furthermore, lumpectomy patients may have trouble accommodating the breast after surgery, due to pain and swelling. The incision site will be incredibly sore and will need support to shower and clean the area correctly to ensure proper healing. Further, a user will need to support the natural weight of the breast to prevent the incision site from pulling and causing pain. Additionally, a user may have increased sensitivity to the water pressure on the incision site. Accordingly, a device that helps users to properly bathe and clean an incision site without severe pain is needed. Further, a device that supports the natural weight of the breast during a shower, is advantageous as well.

Therefore, there exists a long-felt need in the art for a lumpectomy shower bra device that provides users with a modified bra capable of comfortably supporting the breasts while bathing. There is also a long-felt need in the art for a lumpectomy shower bra device that offers a way for lumpectomy patients to bathe independently with minimal pain and irritation. Further, there is a long-felt need in the art for a lumpectomy shower bra device that allows lumpectomy patients to shower without worry of water pressure hitting the incision site and causing severe pain. Moreover, there is a long-felt need in the art for a device that includes soft, lily pad-like cups that supports the breasts. Further, there is a long-felt need in the art for a lumpectomy shower bra device that utilizes quick drying material, such that a user can consistently reuse the bra for daily bathing. Finally, there is a long-felt need in the art for a lumpectomy shower bra device that comprises a closure mechanism to ensure the bra remains secure while bathing.

The subject matter disclosed and claimed herein, in one embodiment thereof, comprises a lumpectomy shower bra device. The device is designed for lumpectomy patients to comfortably and independently shower. Specifically, the lumpectomy shower bra device comprises a racer back style with straps over the shoulder, and a closure positioned in the front of the bra. The device comprises soft, lily pad-like cups which supports a user’s breasts and prevents water pressure from hitting the incision site. Further, the bra device is also constructed using a quick-drying material for daily reuse while bathing. Thus, users can utilize both arms while taking a shower or bathing, and can utilize shampoo, conditioner, body scrubs, etc.

In this manner, the lumpectomy shower bra device of the present invention accomplishes all of the foregoing objectives and provides users with a device that supports a user’s breasts while bathing or showering. The device is configured to include lily pad-like cups that supports the breasts and a closure mechanism that ensures the bra remains secure while bathing. The device helps users to properly bathe and clean the incision site without severe pain.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a lumpectomy shower bra device. The device is designed for lumpectomy patients to comfortably and independently shower. Specifically, the lumpectomy shower bra (or brassiere) device comprises a racer back style bra with straps over the shoulder, and a closure positioned in the front of the bra. The device comprises soft, lily pad-like cups which supports a user’s breasts and prevents water pressure from hitting the incision site, as well as granting the user access to the incision site for cleaning.

The present invention is designed to meet the needs of a patient recovering from breast surgery in general and/or from other surgical procedures. Generally, the lumpectomy shower bra device is designed to be worn while bathing but can be worn as a conventional bra under clothing, if desired as well. In one embodiment, the lumpectomy shower bra device is configured in a racerback style with two straps that are positioned over a user’s shoulder, which then come together in the back to form a rear panel. However, other suitable bra styles can be utilized as is known in the art, such as a bralette, a strapless bra, a balconette bra, a T-shirt bra, a wireless bra, a sports bra, a push-up bra, an un-lined bra, etc., depending on the needs and/or wants of a user, as well as the amount of support a user desires.

In one embodiment, the lumpectomy shower bra device comprises a right and left bra cup component secured together. The bra cup components act to hold and support a user’s breasts or prostheses. Typically, the right and left bra cup components are symmetrical in size and shape, but do not have to be, and depending on the needs and/or wants of a user, can be manufactured in different sizes and shapes. For example, if the user has had a mastectomy on one side, the corresponding bra cup component can be sized accordingly. Further, if the user has had a mastectomy, then the bra cup components of the device, may be sized prior to surgery so that a balanced, womanly look may be achieved immediately after surgery without costly and time-consuming fittings.

More specifically, the shoulder straps of the bra device extend from the top of each bra cup component to the top of an adjacent rear panel. Alternatively, the straps may form a crisscross pattern, wherein the straps extend from the top of each bra cup component to the top of the opposite rear panel, or the straps can be arranged in any suitable configuration as is known in the art. Further, an adjustable tab is provided for adjusting the length of the shoulder straps in accordance with the size of the user. However, it should also be appreciated that the shoulder strap adjustable tabs may be eliminated by the use of an elastic material.

In another embodiment, the right and left bra cup components are configured in a lily pad-like shape, such that a notch, slit, or opening is positioned near the top of a user’s breast, exposing the surgical scar. This lily pad-like shape helps prevent sensitivity to the water pressure on the incision. Accordingly, use of the bra device in the shower, prevents water pressure from directly hitting the incision site and causing pain. Any suitable shape as is known in the art, can be utilized for the bra cup components, as long as a section is left open, exposing the surgical incision site.

Furthermore, the lily pad-like shape also allows users to properly clean and sterilize the incision site, without severe pain. Thus, exposing the surgical scar allows users to be able to correctly wash and clean the surgical incision site, while wearing the bra device in the shower and/or bath, to ensure proper healing. The lily pad-like shape allows a user to comfortably wear a bra post-surgery, without the bra irritating the surgical incision site and causing an infection.

Additionally, the right and left bra cup components offer support for the breasts, preventing the incision site from pulling, causing pain. As conventional bras, the bra cup components support a user’s breasts. Specifically, the bra cup components of the bra device accommodate a user’s breasts post-surgery and act to support the natural weight of the breasts to prevent the incision site from pulling. Typically, a user’s breast(s) is/are sore and swollen after surgery, making it difficult for users to wear a conventional bra, even though the incision site needs support for healing. Accordingly, the bra cup components of the present invention comfortably support a user’s breasts post-surgery, thereby allowing for a shorter healing time.

In one embodiment, one or both of the right and left bra cup components can comprise a pocket for securely receiving and retaining a prosthesis for one or both breasts. The pockets consist of overlapping panels, which allow the prosthesis to securely remain in the desired position and location. Specifically, the pockets are attached to the rear facing portion of bra cup component to retain a prosthesis.

In yet another embodiment, the right and left bra cup components and the rear panel are secured together via a torso band component, or any other suitable securing means as is known in the art. Typically, the torso band component is elasticized to maximize comfort for the user, but can be manufactured of any suitable material as is known in the art. The torso band extends around the lower edge of the bra device to form an almost continuous strip of material from one end of the closure mechanism to the other end of the closure mechanism, and along the lower edge of the bra cup components and the lower edge of the rear panel.

In one embodiment, the lumpectomy shower bra device comprises a front closure, which allows a user to easily hook and unhook the bra device without difficulty, even post-surgery. The front closure also ensures the bra device remains secure, even while bathing and/or showering. The closure component is positioned on the torso band component but does not have to be a front closure and can be positioned in the rear of the bra device, or in any suitable position along the torso band component as is known in the art, depending on the needs and/or wants of a user. For example, in many cases, a mastectomy female encounters difficulty in clasping a bra having a clasp disposed in the rear portion of the chest band. However, other females encounter little or no difficulty in fastening a rear clasp bra.

In another embodiment, the bra device can comprise dual closures in the front and back of the present invention, allowing ease in removing or putting on the bra device, also allowing ease in examination by a physician to the areas surrounding the incision site. Moreover, the front and back closures allow the bra device to be easily placed and positioned on the user post-surgery and further allows the user to adjust the position of the device on her own body at will, without assistance.

Furthermore, a wide choice of fasteners are available for the front and back closures. Typically, Velcro is the preferred material for the front closure, with hook and eyes preferably used for the back closure. However, such preferred materials do not preclude alternative use of Velcro or other hook-and-loop fasteners, zippers, buttons and buttonholes, snaps, hooks and eyes, other types of hook arrangements, etc., as well as any other suitable fasteners as is known in the art.

In yet another embodiment, the lumpectomy shower bra device comprises right and left apertures sized and positioned to receive drain tubes extending from surgically implanted drainage systems. The apertures are positioned so that the drain tubes feed directly into a fluid collection pouch, minimizing the inherent dangers of accidentally pulling or tugging on the drainage assembly.

In another embodiment, the lumpectomy shower bra device comprises a plurality of indicia.

In yet another embodiment, the lumpectomy shower bra device must provide both function and comfort. To that end, a wide range of synthetic materials and natural materials may be used in the manufacture of the lumpectomy shower bra device. For example, synthetic elastomers including Lycra and Spandex may be used, as well as other conventionally known materials in the art. Natural/synthetic blended materials may include cotton, linen, rayon, or ramie. It is most desirable that the materials employed, whether blended or unblended, provide a soft, smooth feel against a user’s skin. Additionally, the lumpectomy shower bra device must also be comprised of a quick-drying material, such that the bra device can be reused daily after bathing. However, all conventionally known materials are deemed to be within the scope of the present invention.

In yet another embodiment, a method of showering or bathing after a lumpectomy while wearing the lumpectomy shower bra device is described. The method includes the steps of securing a lumpectomy shower bra device to a user. The method also comprises fastening the lumpectomy shower bra device via the front closure. Further, the method comprises showering or bathing while wearing the lumpectomy shower bra device. The method comprises washing the incision site while wearing the lumpectomy shower bra device. Finally, once showered, removing the lumpectomy shower bra device and letting it dry for the next use.

In yet another embodiment of the present invention, a lumpectomy shower bra device is disclosed for lumpectomy patients to comfortably and independently shower. Specifically, the lumpectomy shower bra device comprises a racer back style with straps over the shoulder, and a closure positioned in the front of the bra. The device comprises soft, lily pad-like cups which supports a user’s breasts and prevents water pressure from hitting the incision site. Further, the bra device is also constructed using a quick-drying material for daily reuse while bathing. Thus, users can utilize both arms while taking a shower or bathing, and can utilize shampoo, conditioner, body scrubs, etc.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the lumpectomy shower bra device of the present invention disclosing the lily pad-like cups in accordance with the disclosed architecture;

FIG. 2 illustrates a rear perspective view of another potential embodiment of the lumpectomy shower bra device of the present invention disclosing the racer back straps in accordance with the disclosed architecture;

FIG. 3 illustrates a perspective view of another potential embodiment of the lumpectomy shower bra device of the present invention drying quickly after use in accordance with the disclosed architecture;

FIG. 4 illustrates a perspective view showing how one potential embodiment of the lumpectomy shower bra device is worn in accordance with the disclosed architecture;

FIG. 5 illustrates a perspective view disclosing how an incision site can be cleaned while wearing one potential embodiment of the lumpectomy shower bra device of the present invention in accordance with the disclosed architecture; and

FIG. 6 illustrates a flowchart showing the method of showering or bathing after a lumpectomy while wearing the lumpectomy shower bra device in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a lumpectomy shower bra device that provides users with a modified bra capable of comfortably supporting the breasts while bathing. There is also a long-felt need in the art for a lumpectomy shower bra device that offers a way for lumpectomy patients to bathe independently with minimal pain and irritation. Further, there is a long-felt need in the art for a lumpectomy shower bra device that allows lumpectomy patients to shower without worry of water pressure hitting the incision site and causing severe pain. Moreover, there is a long-felt need in the art for a device that includes soft, lily pad-like cups that supports the breasts. Further, there is a long-felt need in the art for a lumpectomy shower bra device that utilizes quick drying material, such that a user can consistently reuse the bra for daily bathing. Finally, there is a long-felt need in the art for a lumpectomy shower bra device that comprises a closure mechanism to ensure the bra remains secure while bathing.

The present invention, in one exemplary embodiment, is a novel lumpectomy shower bra device designed for lumpectomy patients to comfortably and independently shower. Specifically, the lumpectomy shower bra device comprises a racer back style with straps over the shoulder, and a closure positioned in the front of the bra. The device comprises soft, lily pad-like cups which supports a user’s breasts and prevents water pressure from hitting the incision site. Further, the bra device is also constructed using a quick-drying material for daily reuse while bathing. The present invention also includes a novel method of showering or bathing after a lumpectomy while wearing the lumpectomy shower bra device. The method includes the steps of securing a lumpectomy shower bra device to a user. The method also comprises fastening the lumpectomy shower bra device via the front closure. Further, the method comprises showering or bathing while wearing the lumpectomy shower bra device. The method comprises washing the incision site while wearing the lumpectomy shower bra device. Finally, once showered, removing the lumpectomy shower bra device and letting it dry for the next use.

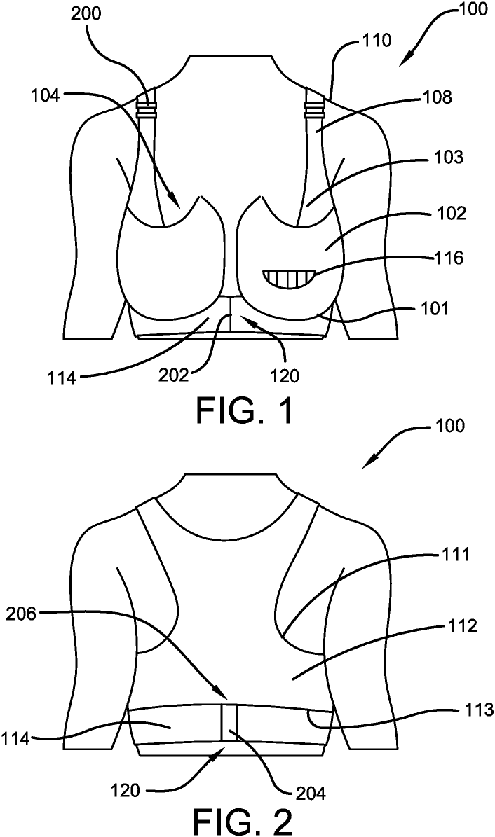

Referring initially to the drawings, FIG. 1 illustrates a perspective view of one embodiment of the lumpectomy shower bra device 100 of the present invention. In the present embodiment, the lumpectomy shower bra device 100 is an improved lumpectomy shower bra device that comfortably supports a user’s breasts while showering or bathing. The device 100 is especially designed to allow a user who has had a lumpectomy, a mastectomy, etc., or any other surgical procedure as is known in the art, or any other suitable user as is known in the art, a way for users to bathe independently with minimal pain and irritation. More specifically, the device 100 comprises soft, lily pad-like bra cup components 102 which supports a user’s breasts 104 and prevents water pressure from hitting the surgical incision site 106, as well as granting access to the incision site 106 for cleaning.

The present invention is designed to meet the needs of a patient recovering from breast surgery in general and/or from other surgical procedures. Generally, the lumpectomy shower bra device 100 is designed to be worn while bathing but can be worn as a conventional bra under clothing, if desired as well. In one embodiment, the lumpectomy shower bra device 100 is configured in a racerback style with two straps 108 that are positioned over a user’s shoulder 110, which then come together in the back to form a rear panel 112. However, other suitable bra styles can be utilized as is known in the art, such as a bralette, a strapless bra, a balconette bra, a T-shirt bra, a wireless bra, a sports bra, a push-up bra, an un-lined bra, etc., depending on the needs and/or wants of a user, as well as the amount of support a user desires.

Furthermore, the lumpectomy shower bra device 100 comprises a right and left bra cup component 102 secured together via a torso band component 114. Specifically, the straps 108 engage the top 103 of the bra cup component 102 and the top 111 of the rear panel 112, connecting them together and the torso band component 114 connects the bottom 101 of the bra cup components 102 and the bottom 113 of the rear panel 112 together into one integral piece. The bra cup components 102 act to hold and support a user’s breast 104 or prosthesis (not shown). Typically, the right and left bra cup components 102 are symmetrical in size and shape, but do not have to be, and depending on the needs and/or wants of a user, can be manufactured in different sizes and shapes. For example, if the user has had a mastectomy on one side, the corresponding bra cup component 102 can be sized accordingly. Further, if the user has had a mastectomy, then the bra cup components 102 of the device 100, may be sized prior to surgery so that a balanced, womanly look may be achieved immediately after surgery without costly and time-consuming fittings.

Additionally, the torso band component 114 which secures the right and left bra cup components 102 and the rear panel 112 together can be a thin, elasticized band to prevent any irritation and/or uncomfortableness to the user. However, any other suitable securing means as is known in the art can be utilized besides the torso band component 114. As stated supra, typically, the torso band component 114 is elasticized to maximize comfort for the user, but can be manufactured of any suitable material as is known in the art. Specifically, the torso band component 114 extends around the lower edge of the bra device 100 to form an almost continuous strip of material from one end of a closure mechanism 120 to the other end of the closure mechanism 120, and along the lower edge (or bottom 101) of the bra cup components 102 and the lower edge (or bottom 113) of the rear panel 112.

In one embodiment, one or both of the right and left bra cup components 102 can comprise a pocket 116 for securely receiving and retaining a prosthesis for one or both breasts 104. The pockets 116 consist of overlapping panels, which allow the prosthesis to securely remain in the desired position and location. Specifically, the pockets 116 are attached to the rear facing portion 118 of the bra cup component 102 to retain a prosthesis.

FIGS. 1-2 illustrate a perspective view of another embodiment of the lumpectomy shower bra device 100 of the present invention. In this embodiment, the shoulder straps 108 of the bra device 100 extend from the top 103 of each bra cup component 102 to the top 111 of an adjacent rear panel 112. Alternatively, the straps 108 may form a crisscross pattern, wherein the straps 108 extend from the top 103 of each bra cup component 102 to the top 111 of the opposite rear panel 112, or the straps 108 can be arranged in any suitable configuration as is known in the art. Further, an adjustable tab 200 is provided for adjusting the length of the shoulder straps 108 in accordance with the size of the user. However, it should also be appreciated that the shoulder strap adjustable tabs 200 may be eliminated by the use of an elastic material.

In one embodiment, the lumpectomy shower bra device 100 comprises a front closure 202, which allows a user to easily hook and unhook the bra device 100 without difficulty, even post-surgery. The front closure 202 also ensures the bra device 100 remains secure, even while bathing and/or showering. The closure component (or mechanism 120) is positioned on the torso band component 114 but does not have to be a front closure 202 and can be positioned in the rear of the bra device 100, or in any suitable position along the torso band component 114 as is known in the art, depending on the needs and/or wants of a user. For example, in many cases, a mastectomy female encounters difficulty in clasping a bra having a clasp disposed in the rear portion of the chest band. However, other females encounter little or no difficulty in fastening a rear clasp bra.

In another embodiment, the bra device 100 can comprise dual closures in the front 202 and back 204 of the present invention, allowing ease in removing or putting on the bra device 100, also allowing ease in examination by a physician to the areas surrounding the incision site 106. Moreover, the front 202 and back 204 closures allow the bra device 100 to be easily placed and positioned on the user post-surgery and further allows the user to adjust the position of the device 100 on her own body at will, without assistance.

Furthermore, a wide choice of fasteners 206 are available for the front 202 and back 204 closures. Typically, Velcro is the preferred material for the front closure 202, with hook and eyes preferably used for the back closure 204. However, such preferred materials do not preclude alternative use of Velcro or other hook-and-loop fasteners, zippers, buttons and buttonholes, snaps, hooks and eyes, other types of hook arrangements, etc., as well as any other suitable fasteners as is known in the art.

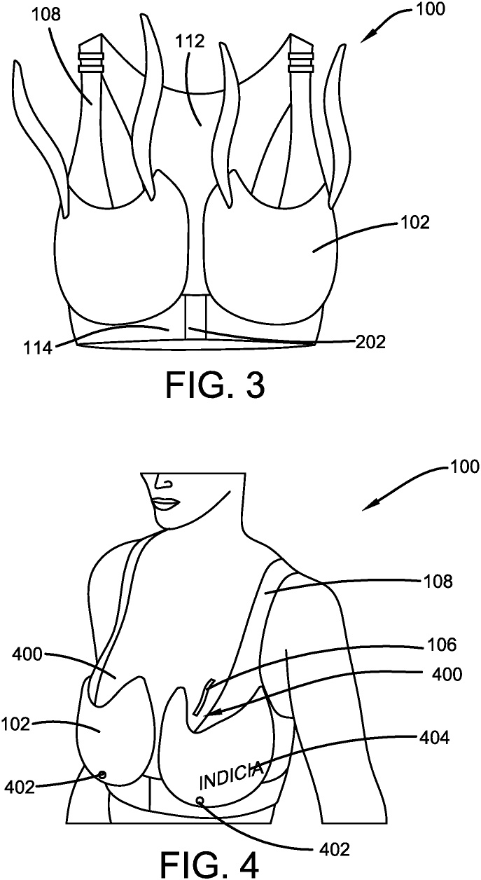

FIG. 3 illustrates a perspective view of another embodiment of the lumpectomy shower bra device 100 of the present invention. The lumpectomy shower bra device 100 must provide both function and comfort. To that end, a wide range of synthetic materials and natural materials may be used in the manufacture of the lumpectomy shower bra device 100. For example, synthetic elastomers including Lycra and Spandex may be used, as well as other conventionally known materials in the art. Natural/synthetic blended materials may include cotton, linen, rayon, or ramie. It is most desirable that the materials employed, whether blended or unblended, provide a soft, smooth feel against a user’s skin. Additionally, the lumpectomy shower bra device 100 must also be comprised of a quick-drying material, such that the bra device 100 can be reused daily after bathing. However, all conventionally known materials are deemed to be within the scope of the present invention.

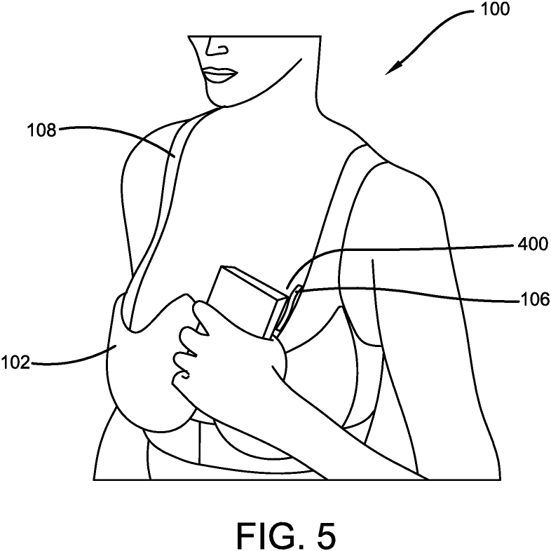

FIGS. 4-5 illustrate a perspective view of another embodiment of the lumpectomy shower bra device 100 of the present invention in use. In this embodiment, the right and left bra cup components 102 are configured in a lily pad-like shape, such that a notch, slit, or opening 400 is positioned near the top of a user’s breast 104, exposing the surgical scar 106. This lily pad-like shape helps prevent sensitivity to the water pressure on the incision 106. Accordingly, use of the bra device 100 in the shower, prevents water pressure from directly hitting the incision site 106 and causing pain. Any suitable shape as is known in the art, can be utilized for the bra cup components 102, as long as a section is left open, exposing the surgical incision site 106.

Furthermore, the lily pad-like shape also allows users to properly clean and sterilize the incision site 106, without severe pain. Thus, exposing the surgical scar 106 allows users to be able to correctly wash and clean the surgical incision site 106, while wearing the bra device 100 in the shower and/or bath, to ensure proper healing. The lily pad-like shape allows a user to comfortably wear a bra post-surgery, without the bra irritating the surgical incision site 106 and causing an infection.

Additionally, the right and left bra cup components 102 offer support for the breasts 104, preventing the incision site 106 from pulling, causing pain. As conventional bras, the bra cup components 102 support a user’s breasts 104. Specifically, the bra cup components 102 of the bra device 100 accommodate a user’s breasts 104 post-surgery and act to support the natural weight of the breasts 104 to prevent the incision site 106 from pulling. Typically, a user’s breast(s) 104 are sore and swollen after surgery, making it difficult for users to wear a conventional bra, even though the incision site 106 needs support for healing. Accordingly, the bra cup components 102 of the present invention comfortably support a user’s breasts 104 post-surgery, thereby allowing for a shorter recovery period.

In yet another embodiment, the lumpectomy shower bra device 100 comprises right and left apertures 402 sized and positioned to receive drain tubes (not shown) extending from surgically implanted drainage systems. The apertures 402 are positioned so that the drain tubes feed directly into a fluid collection pouch, minimizing the inherent dangers of accidentally pulling or tugging on the drainage assembly.

In another embodiment, the lumpectomy shower bra device 100 comprises a plurality of indicia 404. The bra cup components 102 and/or the torso band component 114 of the lumpectomy shower bra device 100 may include advertising, a trademark, or other letters, designs, or characters, printed, painted, stamped, or integrated into the components 102 and 114, or any other indicia 404 as is known in the art. Specifically, any suitable indicia 404 as is known in the art can be included, such as but not limited to, patterns, logos, emblems, images, symbols, designs, letters, words, characters, animals, advertisements, brands, etc., that may or may not be brassiere, surgical or brand related.

As shown in FIG. 5, a lumpectomy shower bra device 100 is disclosed for lumpectomy patients to comfortably and independently shower. Specifically, the lumpectomy shower bra device 100 comprises a racer back style with straps 108 over the shoulder 110, and a closure mechanism 120 positioned in the front 202 of the bra device 100. The device 100 comprises soft, lily pad-like bra cup components 102 which support a user’s breasts 104 and prevent water pressure from hitting the incision site 106. Further, the bra device 100 is also constructed using a quick-drying material for daily reuse while bathing. Thus, users can utilize both arms while taking a shower or bathing, and can utilize shampoo, conditioner, body scrubs, etc.

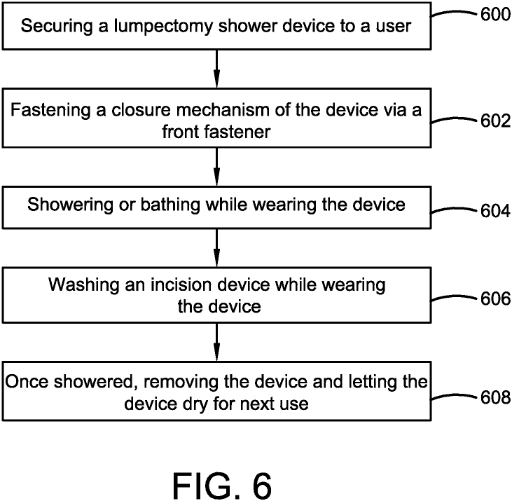

FIG. 6 illustrates a flowchart of the method of showering or bathing after a lumpectomy while wearing the lumpectomy shower bra device. The method includes the steps of at 600, securing a lumpectomy shower bra device to a user. The method also comprises at 602, fastening the closure mechanism of the lumpectomy shower bra device via the front closure. Further, the method comprises at 604, showering or bathing while wearing the lumpectomy shower bra device. The method comprises at 606, washing the incision site while wearing the lumpectomy shower bra device. Finally, once showered, at 608, removing the lumpectomy shower bra device and letting it dry for the next use.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “lumpectomy shower bra device”, “bra device”, and “device” are interchangeable and refer to the lumpectomy shower bra device 100 of the present invention.

Notwithstanding the foregoing, the lumpectomy shower bra device 100 of the present invention can be of any suitable size and configuration as is known in the art without affecting the overall concept of the invention, provided that it accomplishes the above-stated objectives. One of ordinary skill in the art will appreciate that the lumpectomy shower bra device 100 as shown in FIGS. 1-6 is for illustrative purposes only, and that many other sizes and shapes of the lumpectomy shower bra device 100 are well within the scope of the present disclosure. Although the dimensions of the lumpectomy shower bra device 100 are important design parameters for user convenience, the lumpectomy shower bra device 100 may be of any size that ensures optimal performance during use and/or that suits the user’s needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A lumpectomy shower bra device that comfortably supports a user’s breasts while showering or bathing, the lumpectomy shower bra device comprising:
a pair of bra cup components;
a rear panel;
a pair of straps that secure a top of the pair of bra cup components to a top of the rear panel; and
a torso band component that secures a bottom of the pair of bra cup components to a bottom of the rear panel, wherein the pair of bra cup components are shaped to expose a user’s surgical incision site to prevent a water pressure from hitting the user’s surgical incision site but still granting the user access to clean the user’s surgical incision site.

2. The lumpectomy shower bra device of claim 1, wherein the pair of straps are positioned over a user’s shoulder and then come together in a racerback configuration.

3. The lumpectomy shower bra device of claim 2, wherein the pair of bra cup components are symmetrical in both size and shape.

4. The lumpectomy shower bra device of claim 1, wherein the torso band component is comprised of an elastic material.

5. The lumpectomy shower bra device of claim 4, wherein the torso band component comprises a front closure.

6. The lumpectomy shower bra device of claim 4, wherein the torso band component comprises a back closure.

7. The lumpectomy shower bra device of claim 4, wherein the torso band component comprises both a front closure and a back closure.

8. The lumpectomy shower bra device of claim 1, wherein at least one of the pair of bra cup components comprises a pocket for retaining a prosthesis.

9. The lumpectomy shower bra device of claim 1, wherein the pair of straps comprise an adjustable tab for adjusting a length of the pair of straps.

10. The lumpectomy shower bra device of claim 1, wherein the pair of straps are comprised of an elastic material.

11. The lumpectomy shower bra device of claim 1, wherein the lumpectomy shower bra device is comprised of a quick-drying material.

12. The lumpectomy shower bra device of claim 1, wherein the pair of bra cup components are manufactured in a lily pad-like shape, such that an opening is provided at a top of the user’s breast to expose the user’s surgical incision site.

13. The lumpectomy shower bra device of claim 1 further comprising right and left apertures sized and positioned to receive a user’s drain tubes post-surgery.

14. A lumpectomy shower bra device that comfortably supports a user’s breasts while showering or bathing, the lumpectomy shower bra device comprising:
a pair of lily pad-like shaped bra cup components to support the user’s breasts;
a rear panel;
a pair of straps positioned over a user’s shoulder and that come together in a racerback configuration, wherein the pair of straps secure a top of the pair of lily pad-like shaped bra cup components to a top of the rear panel; and
an elastic torso band component with a front closure for securing the lumpectomy shower bra device to a user, wherein the elastic torso band component secures a bottom of the pair of lily pad-like shaped bra cup components to a bottom of the rear panel, and further wherein the pair of lily pad-like shaped bra cup components are shaped to expose a user’s surgical incision site to prevent a water pressure from hitting the user’s surgical incision site while still granting the user access to clean the user’s surgical incision site.

15. The lumpectomy shower bra device of claim 14 further comprising a plurality of indicia.

16. The lumpectomy shower bra device of claim 14, wherein at least one of the pair of lily pad-like shaped bra cup components comprises a pocket for retaining a prosthesis.

17. The lumpectomy shower bra device of claim 14, wherein the pair of straps comprise an adjustable tab for adjusting a length of the pair of straps.

18. The lumpectomy shower bra device of claim 14, wherein the pair of straps are comprised of an elastic material.

19. The lumpectomy shower bra device of claim 14 further comprising a right aperture and a left aperture sized and positioned to receive a user’s drain tubes post-surgery.

20. A method of showering or bathing after a lumpectomy while wearing a lumpectomy shower bra device, the method comprising the steps of:
securing the lumpectomy shower bra device to a user;
fastening a closure mechanism of the lumpectomy shower bra device via a front closure;
showering or bathing while wearing the lumpectomy shower bra device;
washing an incision site while wearing the lumpectomy shower bra device; and
removing the lumpectomy shower bra device once showered, and letting the lumpectomy shower bra device dry for a next use.
